(19) United States
(12) Patent Application Publication
(10) Pub. No.: US 2017/0281705 A1
(43) Pub. Date: Oct. 5, 2017

(54) ANTI-VIRAL THERAPEUTIC COMPOSITIONS

(71) Applicant: CAPE KINGDOM NUTRACEUTICALS (PTY) LTD, Big Bay (ZA)

(72) Inventor: Michael Duncombe STANDER, Big Bay (ZA)

(73) Assignee: CAPE KINGDOM NUTRACEUTICALS (PTY) LTD, Big Bay (ZA)

(21) Appl. No.: 15/532,436

(22) PCT Filed: Dec. 1, 2015
(86) PCT No.: PCT/IB2015/059236

§ 371 Date: Jun. 1, 2017

(30) Foreign Application Priority Data
Dec. 1, 2014 (ZA) 2014/08790

(51) Int. Cl. A61K 36/75; A61K 45/06

(52) U.S. Cl. CPC A61K 36/75; A61K 45/06; A61K 2236/55

(57) ABSTRACT

The invention discloses an anti-viral therapeutic composition, which includes at least one anti-viral active ingredient originating from an aqueous Buchu extract or bio-active fraction thereof in a pharmaceutically acceptable form. The composition can be a pharmaceutical composition including a therapeutically effective amount of at least one or more anti-viral active ingredient and one or more pharmaceutically acceptable carriers or additives. The aqueous Buchu extract is obtained from the species Agasthoma betulina (round-leaf Buchu) and/or Agathosma crenulata (oval-leaf Buchu).

### FIELD OF INVENTION

The present invention relates to anti-viral therapeutic composition. More particularly, the present invention relates to anti-viral therapeutic compositions of Buchu plant material extracts.

### BACKGROUND TO INVENTION

Buchu is one of the best known medicinal plants of South Africa and is indigenous to the Cedarberg Mountains and surrounding areas. Despite its popularity little scientific evidence exists about the various medicinal uses of this small fynbos shrub from the family Rustaceae. The two primary species of Buchu used commercially are Agasthoma betulina (round-leaf Buchu) and Agathosma crenulata (oval-leaf Buchu). Besides its medicinal properties, Buchu oil is also used in the flavourant and fragrance industry, currently the largest commercial use thereof.

Buchu oil is typically prepared in a (high vacuum) low steam distillation process in which the Buchu oil required for the commercial market is extracted from the plant material and separated from the by-products of this steam distillation process.

It is an object of the invention to suggest an anti-viral therapeutic compositions of Buchu plant material extracts.

### SUMMARY OF INVENTION

According to the invention, there is provided an anti-viral therapeutic composition comprising at least one anti-viral active ingredient originating from an aqueous Buchu extract or bio-active fraction thereof in a pharmaceutically acceptable form.

Preferably, the anti-viral therapeutic composition is a pharmaceutical composition comprising a therapeutically effective amount of at least one or more anti-viral active ingredient and one or more pharmaceutically acceptable carriers or additives.

The invention extends to a modified aqueous Buchu extract or bio-active fraction thereof comprising an effective amount of one or more anti-viral active ingredients.

The anti-viral active ingredient is preferably an anti-HIV molecule or component of an aqueous Buchu extract or bio-active fraction thereof.

The invention also extends to a therapeutic composition, pharmaceutical composition or modified aqueous Buchu extract or bio-active fraction thereof for use in a method of inducing an anti-viral response, in particular an anti-HIV response, in a mammal, preferably a human, in need thereof.

The invention extends further to the use of an aqueous Buchu extract or bio-active fraction thereof in the manufacture of a medicament for use in a method of inducing an anti-viral response, in particular an anti-HIV response, in a mammal, preferably a human, in need thereof.

According to a further aspect of the invention, there is provided a method of treating a viral infection such as a HIV infection comprising administering to a patient in need thereof a therapeutically effective amount of at least one active ingredient obtained from an aqueous Buchu extract or bio-active fraction thereof.

The aqueous Buchu extract may be obtained from the species Agasthoma betulina (round-leaf Buchu) and/or Agathosma crenulata (oval-leaf Buchu).

### BRIEF DESCRIPTION OF DRAWINGS

The invention will now be described by way of example with reference to the accompanying schematic drawings.

In the drawings there is shown in:

FIGS. 1a/1b: graphs of the anti-viral activity of round leaf and oval leaf aqueous Buchu extracts of the invention; and

FIGS. 2a/2b: graphs of the effect of dilution on the anti-viral activity of the round leaf and oval leaf aqueous Buchu extracts of the invention.

### DETAILED DESCRIPTION OF DRAWINGS

Experiments have shown that the water soluble molecules or components of Buchu plant material show anti-viral activity, particularly anti-HIV activity.

In order to test the anti-viral activity of the water soluble components of Buchu plant material, the aqueous portion was obtained from a high vacuum low steam distillation process traditionally used for extracting Buchu oil from Buchu plant material. The aqueous portion was separated from the oil portion, and treated in order to remove impurities and other non-water-soluble components, including Buchu oil residue. This modified aqueous Buchu extract is hereinafter referred to as “Buchu water” for convenience.

For testing purposes separate round-leaf and oval-leaf Buchu water samples were tested to identify the anti-viral activity of each, particularly against HIV.

The separate test samples were prepared by back extracting round leaf and oval leaf Buchu water, respectively, using chloroform (or an equivalent solvent) and concentrated under reduced pressure (DURPed) by rotary evaporation. The residual liquids that remained after all the chloroform had been removed were subsequently dissolved in pure Methanol to a final volume of 1:10 of the respective original water volume (hence considered as a 10× concentrated form).

A human T cell line (5.25) was used as host for the HIV replication. A known amount of virus (HIV-1) was added to these continuously growing cells and the test sample under investigation was added simultaneously. Following an incubation of 3 hours, the cells were washed to remove non-bound virus and excess test sample. The cells were re-incubated with fresh medium and monitored for signs of viral replication. This viral replication can be seen microscopically (as cells become large and undergo fusion with each other) or by removing the cell supernatants and measuring the release of an HIV-specific protein called p24. The latter method of determining viral replication (or inhibition thereof) is obviously quantitative and less biased, and was favoured for this investigation.

In order to measure the ability of the Buchu water test samples to inhibit (or not) the HIV-1 innoculum used, the supernatants of the cells used were harvested and the levels of p24 (the core protein of the virus) was measured by ELISA (Enzyme-linked immunosorbant assay). The results are expressed as pg/ml supernatant.

The measurement of p24 production was determined over 2 time periods of culture, namely, 4 and 7 days. The different times selected were based on the possibility that the product under investigation could inhibit the early replication cycles of the virus and that this inhibition could wane after longer incubation periods. The results were normalised to a “positive control”, which are cell cultures that only received the virus inoculum and no test sample (this would represent the 100% value of p24 measured). Likewise, a blank was included where the cells received an equivalent volume of methanol only to account for any inhibitory effects that the solvent may have (referred to as the “blank culture”. We expect little if any interference of the viral replication in the presence of the methanol only. The “negative control” cultures are cells that were cultured for the same periods of time but these received neither the virus inoculum nor the sample under investigation. These cells should therefore produce no significant amounts of p24 as measured by ELISA.

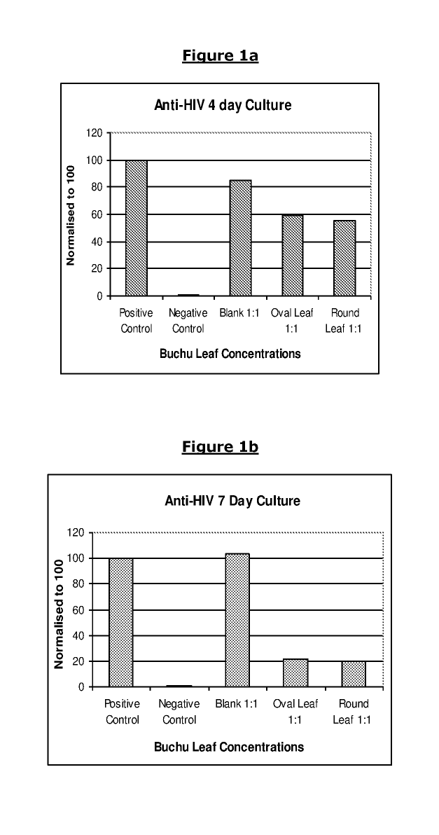

The results of these tests are shown graphically in accompanying FIGS. 1a and 1b. From these graphs it is clear that a 50% (1:1 dilution) of the back-extracted Buchu water was able to inhibit the viral replication after 4 days as well as after 7 days in culture. The 7 day culture appears to be more inhibited compared to the 4 day culture. Without wishing to be bound by theory, early indications are that this represents a slow acting molecule or component that is better at controlling the viral replication long term. This would also tend to indicate that the bio-active molecule(s) or component(s) acts more akin to a protease inhibitor rather than a molecule or compound that prevents or inhibits binding to the host cell. The inhibition of the early replication cycle can be induced by compounds or drugs which inhibit either the initial binding of the virus to the cell membrane or the enzyme Reverse Transcriptase which is coded for by the virus itself. If this enzyme is inhibited, then the synthesis of a complimentary DNA strand based on the virus RNA would be inhibited and the subsequent integration of this cDNA into the host's genetic material would be prevented.

Since our results indicate that the virus is better inhibited at day 7 of culture, this would imply that a later step in the virus cycle is possibly inhibited: this could mean either the inhibition of protease enzymes that are used for viral release from the host cell is inhibited or that the assembly of new viral particles within the cytoplasm of the cell is prevented.

Interestingly, both water preparations exhibited the same activity and the inhibition was not limited to any one species. Both species inhibited the replication of the virus by +/−80% by day 7 of culture . In order to test the effect of dilution on the ability of the round leaf and oval leaf Buchu water, respective samples were made as 1:3 dilutions and tested in the same manner as described above.

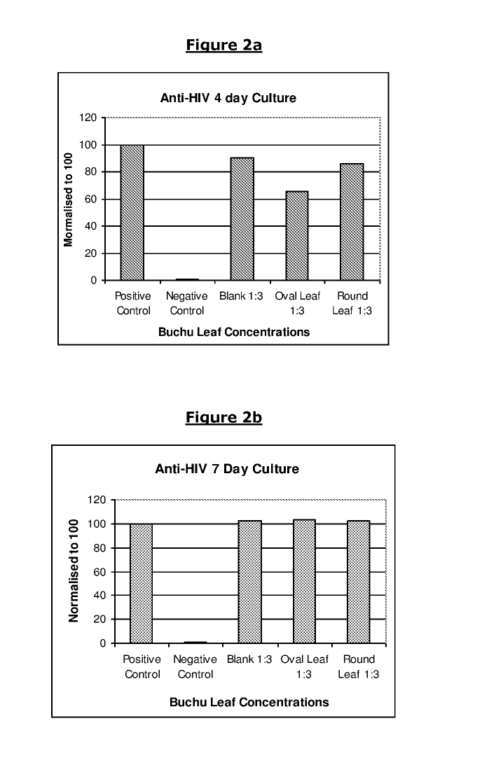

As can be seen from accompanying FIGS. 2a and 2b, the inhibition began to wane once the preparations were diluted at a 1:3 dilution. The effect was less pronounced although some anti-viral activity was still shown. This was especially true for the oval leaf water extract. This implies that the bio-active molecule(s) are present at low concentrations in the current preparations tested and that these require further enrichment.

It is clear that Buchu water extracts prepared from round and oval leaf species contain bio-active molecules able to inhibit HIV viral replication in vitro.

1. An anti-viral therapeutic composition, which includes at least one anti-viral active ingredient originating from an aqueous Buchu extract or bio-active fraction thereof in a pharmaceutically acceptable form.

2. A composition as claimed in claim 1, which is a pharmaceutical composition including a therapeutically effective amount of at least one or more anti-viral active ingredient and one or more pharmaceutically acceptable carriers or additives.

3. A composition as claimed in claim 1, in which the anti-viral active ingredient is preferably an anti-HIV molecule or component of an aqueous Buchu extract or bio-active fraction thereof.

4. A composition as claimed in claim 1, in which the aqueous Buchu extract is obtained from the species Agasthoma betulina (round-leaf Buchu) and/or Agathosma crenulata (oval-leaf Buchu).

5. A modified aqueous Buchu extract or bio-active fraction thereof, which includes an effective amount of one or more anti-viral active ingredients.

6. An extract as claimed in claim 5, in which the anti-viral active ingredient is preferably an anti-HIV molecule or component of an aqueous Buchu extract or bio-active fraction thereof.

7. An extract as claimed in claim 5, in which the aqueous Buchu extract is obtained from the species Agasthoma betulina (round-leaf Buchu) and/or Agathosma crenulata (oval-leaf Buchu).

8. A therapeutic composition, pharmaceutical composition or modified aqueous Buchu extract or bio-active fraction thereof for use in a method of inducing an anti-viral response, in particular an anti-HIV response, in a mammal, preferably a human, in need thereof.

9. A composition as claimed in claim 8, in which the anti-viral active ingredient is preferably an anti-HIV molecule or component of an aqueous Buchu extract or bio-active fraction thereof.

10. A composition as claimed in claim 8, in which the aqueous Buchu extract is obtained from the species Agasthoma betulina (round-leaf Buchu) and/or Agathosma crenulata (oval-leaf Buchu).

11-13. (canceled)

14. A method of treating a viral infection such as a HIV infection, which includes the step of administering to a patient in need thereof a therapeutically effective amount of at least one active ingredient obtained from an aqueous Buchu extract or bio-active fraction thereof.

15. A method as claimed in claim 14, in which the anti-viral active ingredient is preferably an anti-HIV molecule or component of an aqueous Buchu extract or bio-active fraction thereof.

16. A method as claimed in claim 14, in which the aqueous Buchu extract is obtained from the species Agasthoma betulina (round-leaf Buchu) and/or Agathosma crenulata (oval-leaf Buchu).

17-22. (canceled)
